(19) United States
(12) Patent Application Publication
(10) Pub. No.: US 2023/0146484 A1
(43) Pub. Date: May 11, 2023

(54) MICROBIAL HOST CELLS FOR PRODUCTION OF STEVIOL GLYCOSIDES

(71) Applicant: MANUS BIO INC., Cambridge, MA (US)

(72) Inventors: Ryan PHILIPPE, Somerville, MA (US); Ajikumar PARAYIL KUMARAN, Watertown, MA (US); Christine Nicole S. SANTOS, Newton, MA (US)

(21) Appl. No.: 17/981,671

(22) Filed: Nov. 7, 2022

Related U.S. Application Data
(63) Continuation of application No. 16/765,196, filed on May 19, 2020, now abandoned.
(60) Provisional application No. 62/588,646, filed on Nov. 20, 2017.

(51) Int. Cl. C12P 19/56

(52) U.S. Cl. CPC C12P 19/56; C12N 1/205; C12Y 114/14; C12Y 114/13079; C12Y 204/01; C12Y 402/03019

(57) ABSTRACT

The present invention provides engineered cells and methods for making high purity steviol glycosides, including RebM. In some aspects, the present invention provides host cells, such as bacterial cells (including but not limited to E. coli), that are engineered to overexpress and/or delete or inactivate one or more steviol glycoside transport proteins. The bacterial cells selectively export RebM, or other specific combination of steviol glycosides, out of the cell to increase productivity and reduce production costs associated with downstream purification. Non-target steviol glycosides are not transported to the extracellular medium in significant amounts.

### FIELD OF TECHNOLOGY

The present technology relates generally to microbial cells having engineered expression of steviol glycoside transport proteins.

### BACKGROUND

High intensity sweeteners possess a sweetness level that is many times greater than the sweetness level of sucrose. They are essentially non-caloric and are commonly used in diet and reduced-calorie products, including foods and beverages. High intensity sweeteners do not elicit a glycemic response, making them suitable for use in products targeted to diabetics and others interested in controlling their intake of carbohydrates.

Steviol glycosides are a class of compounds found in the leaves of Stevia rebaudiana Bertoni, a perennial shrub of the Asteraceae (Compositae) family native to certain regions of South America. They are characterized structurally by a single base, steviol, differing by the presence of carbohydrate residues at positions C13 and C19. They accumulate in Stevia leaves, composing approximately 10% to 20% of the total dry weight. On a dry weight basis, the four major glycosides found in the leaves of Stevia typically include stevioside (9.1%), rebaudioside A (3.8%), rebaudioside C (0.6-1.0%) and dulcoside A (0.3%). Other known steviol glycosides include rebaudioside B, C, D, E, F and M, steviolbioside and rubusoside.

The minor glycosylation product rebaudioside (RebM) is estimated to be about 200-350 times more potent than sucrose, and is described as possessing a clean, sweet taste with a slightly bitter or licorice aftertaste. Prakash I. et al., Development of Next Generation Stevia Sweetener: Rebaudioside M, Foods 3(1), 162-175 (2014).

RebM, as well as other steviol glycosides, are of great interest to the global food industry, and thus cost effective methods for their production at high yield and purity are desired.

### BRIEF DESCRIPTION OF THE DRAWINGS

FIG. 1 shows the chemical structure of Rebaudioside M (RebM), a minor component of the steviol glycoside family, and which is a derivative of the diterpenoid steviol (box) with six glucosyl-modification groups.

FIG. 2 shows the product titer of RebM in comparison to steviol and other glycosylation products in a representative bioreactor culture expressing an engineered E. coli strain (as described in WO20161/073740A1).

FIG. 3 shows that the majority of RebM accumulates extracellularly. Panel A shows the titer of RebM and steviol glycosides inside and outside of the cell. Panel B shows the same data as the percent of each compound observed extracellularly.

FIG. 4 shows an exemplary pathway for steviol glycoside production, from the steviol core to RebM. Various intermediates and side products are shown. Arrows are known UGT activities with the specific glycosylation activity listed after the UGT prefix (i.e., c13, c19, 1-2, or 1-3), with no reference to relative activity.

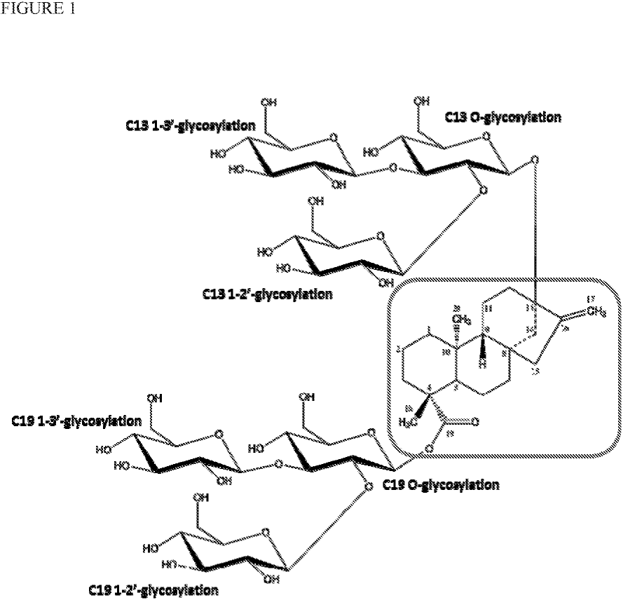

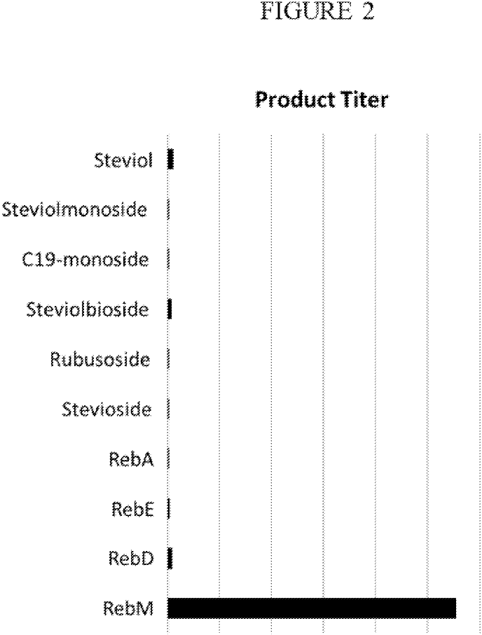

### DETAILED DESCRIPTION

The present invention provides engineered cells and methods for making high purity steviol glycosides, including RebM. In some aspects, the present invention provides host cells, such as bacterial cells (including but not limited to E. coli), that are engineered to overexpress and/or delete or inactivate one or more steviol glycoside transport proteins. The microbial cells selectively export RebM, or other specific combination of steviol glycosides, out of the cell to increase productivity and reduce production costs associated with downstream purification. Non-target steviol glycosides are not transported to the extracellular medium in significant amounts.

Engineering host strains to transport the majority of RebM product (or other steviol glycoside or steviol glycoside combination) out of the cell is very valuable, since it significantly decreases the cost of producing the target steviol glycoside(s) via fermentation. The secretion of the product into the extracellular broth obviates the need for cell lysis and extraction, which reduces both the number of downstream unit operations and the amount of reagents required for recovering the product. In fact, other microbial processes, including those employing yeast, require cellular disruption and additional chromatographic purification methods to separate product from intracellular contaminants derived from the cell lysate.

Accordingly, the present invention provides bacterial cells and methods for making a target steviol glycoside composition, such as RebM, at high purity. In embodiments, the method comprises culturing an engineered a host cell producing one or more target steviol glycosides, wherein the engineered cell comprises recombinant expression of one or more transport proteins that transport the target steviol glycosides into the extracellular medium, and recovering the target steviol glycosides from the extracellular medium. In various embodiments, the cell is a bacterial cell. In other embodiments, the cell is a yeast.

As used herein, the term “engineered” when used with reference to cells means that the cell expresses one or more genes that are not present in their native (non-recombinant) form. That is, the gene may be heterologous or otherwise mutated from its native form, or may be over or under expressed by virtue of non-native expression control sequences. In some embodiments, genes are overexpressed by introducing recombinant genes into the host strain. In other embodiments, the endogenous genes can be overexpressed by modifying, for example, the endogenous promoter or ribosomal binding site. When introducing recombinant genes, the genes may optionally comprise one or more beneficial mutations that improve the specificity of the transport activity (e.g., improve specificity for RebM over RebD). Recombinant enzymes can be expressed from a plasmid or the encoding genes may be integrated into the chromosome, and can be present in single or multiple copies, in some embodiments, for example, about 2 copies, about 5 copies, or about 10 copies per cell.

Various strategies can be employed for engineering the expression or activity of recombinant genes and enzymes, including, for example, modifications or replacement of promoters of different strengths, modifications to the ribosome binding sequence, modifications to the order of genes in an operon or module, gene codon usage, RNA or protein stability, RNA secondary structure, and gene copy number, among others.

In some embodiments, endogenous genes are edited, as opposed to gene complementation. Editing can modify endogenous promoters, ribosomal binding sequences, or other expression control sequences, and/or in some embodiments modifies trans-acting and/or cis-acting factors in gene regulation. Genome editing can take place using CRISPR/Cas genome editing techniques, or similar techniques employing zinc finger nucleases and TALENs. In some embodiments, the endogenous genes are replaced by homologous recombination.

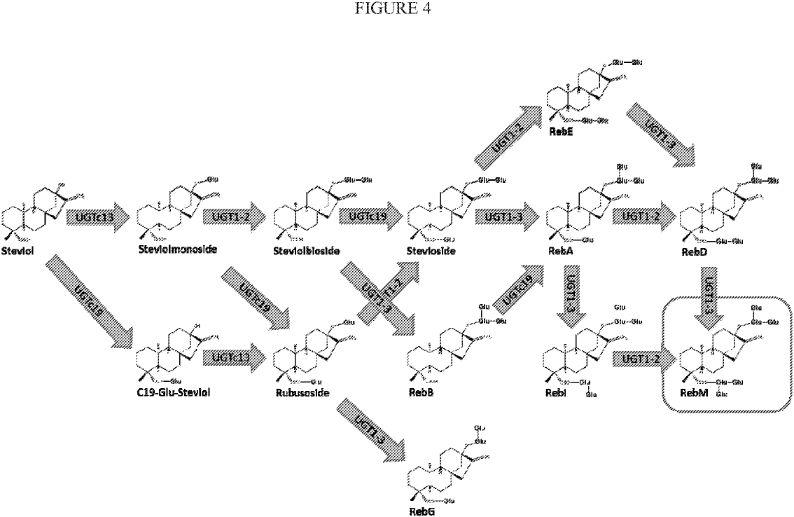

The invention provides for control of the secretion of specific steviol glycosides, such as the selective export of RebM. Maintaining a high ratio of RebM/(all other glycosides) is important for reducing costs associated with the chromatographic separation of unwanted off-pathway byproducts. Specifically, having a high ratio of RebM/RebD (the immediate precursor, FIG. 4) is a key parameter, since separating these two products requires costly preparative and process chromatography, which still fails to deliver a pure RebM product. Thus, the presence of RebD must be minimized in some embodiments to provide a cost-effective process for high-purity RebM.

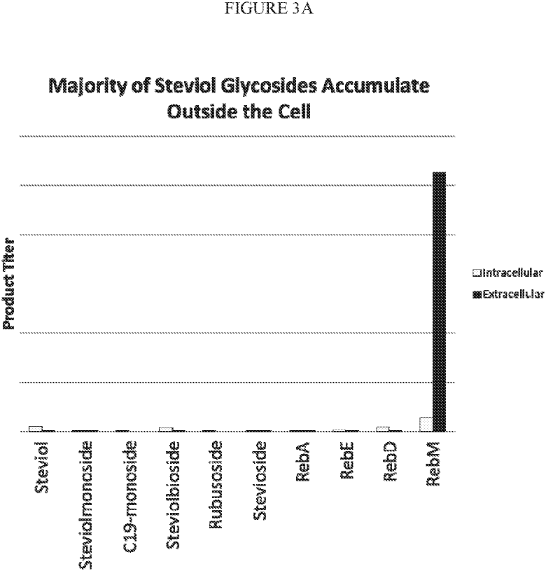

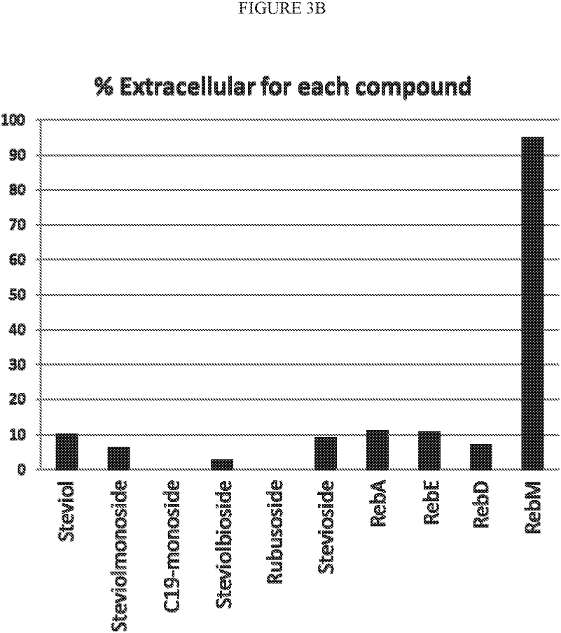

WO 2016/073740, which is hereby incorporated by reference in its entirety, demonstrates that an engineered E. coli strain containing the complete RebM biosynthetic pathway could produce and secrete most of the RebM out of the cell, resulting in a high-purity extracellular RebM product. However, as shown in FIG. 3, appreciable amounts of steviol, steviolmonoside, steviolbioside, stevioside, RebA, RebD, and RebE are also present in the extracellular medium. In accordance with embodiments of this disclosure, a host cell (such as a bacterial cell, e.g., E. coli) is engineered to alter expression of one or more endogenous steviol glycoside transporters and/or complement with one or more heterologous steviol glycoside transporters, to create a host strain capable of high-titer, high purity RebM production, with the majority of product accumulating outside of the cell. The extracellular accumulation of product decreases purification costs and improves ti ter.

In some embodiments, at least 90% of the extracellular steviol glycoside product is the desired or “target” steviol glycoside or combination thereof. In some embodiments, at least 91%, 92%, 93%, 94%, 95%, 96%, 97%, 98%, or 99% of the extracellular steviol glycoside product is the target steviol glycoside or combination. In some embodiments, the desired product consists of RebM. In some embodiments, the RebM:RebD ratio of the extracellular product is greater than 10:1 or greater than 50:1 or greater than 100:1 or greater than 200:1 or greater than 500:1.

In some embodiments, the target steviol glycoside includes one or more selected from steviolmonoside, steviolbioside, rubusoside, dulcoside B, dulcoside A, stevioside, rebaudioside A (RebA), rebaudioside B (RebB), rebaudioside C (RebC), rebaudioside D (RebD), rebaudioside D2 (RebD2), rebaudioside E (RebE), rebaudioside F (RebF), rebaudioside G (RebG), rebaudioside H (RebH), rebaudioside I (RebI), rebaudioside J (RebJ), rebaudioside K (RebK), rebaudioside L (RebL), rebaudioside M (RebM), rebaudioside M2 (RebM2), rebaudioside N (RebN), and rebaudioside O (RebO). In such embodiments, the expression profile of transporters (including deleted, overexpressed, or underexpressed) exports the desired steviol glycoside profile.

The bacterial cell may be a species selected from Escherichia spp., Bacillus spp., Corynebacterium spp., Rhodobacter spp., Zymomonas spp., Vibrio spp., or Pseudomonas spp. For example, the bacterial species may be Escherichia coli, Bacillus subtillus, Corynebacterium glutamicum, Rhodobacter capsulatus, Rhodobacter sphaeroides, Zymomonas mobilis, Vibrio natriegens, or Pseudomonas putida. In some embodiments, the bacterial species is E. coli.

In some embodiments, where the host cell is a eukaryotic cell, the host cell may be a species of Saccharomyces, Pichia, or Yarrowia, including Saccharomyces cerevisiae, Pichia pastoris, and Yarrowia lipolytica.

The bacterial host cell may contain a deletion or inactivaction of one or more endogenous transporters that transport a steviol glycoside other than a target steviol glycoside. Accordingly, transporters that specifically transport the target steviol glycoside (such as RebM) are overexpressed, and transporters that have appreciable affinity for non-target products (such as RebD) may be deleted or inactivated, or underexpressed.

In some embodiments, the bacterial host cell overexpresses one or more bacterial or endogeous transport proteins that transport the target steviol glycoside(s). For example, the transporter may be from the host species, or another bacterial species, and may be engineered to adjust its affinity for the target steviol glycoside over non-target products (e.g., RebM over RebD). For example, the bacterial host cell may overexpress a bacterial or endogenous transporter that is at least 50% identical to an E. coli transporter selected from ampG, araE, araJ, bcr, cynX, emrA, emrB, emrD, emrE, emrK, emrY, entS, exuT, fsr, fucP, galP, garP, glpT, gudP, gudT, hcaT, hsrA, kgtP, lacY, lgoT, lpiT, IptA, lptB, lptC, lptD, lptE, lptF, lptG, mdfA, mdtD, mdtG, mdtH, mdtM, mdtL, mhpT, msbA, nanT, nark, narU, nepI, nimT, nupG, proP, setA, setB, setC, shiA, tfaP, tolC, tsgA, uhpT, xapB, xylE, yaaU, yajR, ybjJ, ycaD, ydeA, ydeF, ydfJ, ydhC, ydhP, ydjE, ydjK, ydiM, ydiN, yebQ, ydcO, yegT, yfaV, yfcJ, ygaY, ygcE, ygcS, yhhS, yhjE, yhjX, yidT, yihN, yjhB, and ynfM. In some embodiments, the endogenous or bacterial transporter is at least about 60%, at least about 70%, at least about 80%, at least about 90%, or at least about 95% identical to the E. coli transporter.

In some embodiments, the bacterial host cell overexpresses an endogenous or bacterial transport protein that is at least 50% identical (at least about 60%, at least about 70%, at least about 80%, at least about 90%, or at least about 95%) identical to an E. coli transporter selected from emrA, emrB, emrK, emrY, lptA, lptB, lptC, lptD, lptE, lptF, lptG, msbA, setA, setB, setC, and tolC. In some embodiments, bacterial host cell overexpresses an endogenous or bacterial transport protein that is at least 50% identical to an E. coli transporter selected from setA, setB, and setC.

In some embodiments, the bacterial cell expresses a transport protein that is at least 50% identical to a transporter from a eukaryotic cell, such as a yeast, fungus, or plant cell. In some embodiments, the transport protein is an ABC family transporter, and which is optionally of a subclass PDR (pleiotropic drug resistance) transporter, MDR (multidrug resistance) transporter, MFS family (Major Facilitator Superfamily) transporter, or SWEET (aka PQ-loop, Saliva, or MtN3 family) family transporter. In other embodiments, the transport protein is of a family selected from: AAAP, SulP, LCT, APC, MOP, ZIP, MPT, VIC, CPA2, ThrE, OPT, Trk, BASS, DMT, MC, AEC, Amt, Nramp, TRP-CC, ACR3, NCS1, PiT, ArsAB, IISP, GUP, MIT, Ctr, and CDF.

In some embodiments, the transporter is an ABC family transport protein (a/k/a ATP-binding cassette transporters), which generally include multiple subunits, one or two of which are transmembrane proteins and one or two of which are membrane-associated ATPases. The ATPase subunits utilize the energy of adenosine triphosphate (ATP) binding and hydrolysis to energize the translocation of various substrates across membranes, either for uptake or for export of the substrate. The ABC family transporter may be of any subclass, including, but not limited to: ABCA, ABCB, ABCC, ABeD, ABCE, ABCF, and ABCG.

In some embodiments, the transport protein is an MFS family transport protein (a/k/a Major Facilitator Superfamily), which are single-polypeptide secondary carriers capable of transporting small solutes in response to chemiosmotic ion gradients. Compounds transported by MFS transport proteins can include simple sugars, oligosaccharides, inositols, drugs, amino acids, nucleosides, organophosphate esters, Krebs cycle metabolites, and a large variety of organic and inorganic anions and cations. By way of example, MFS transport proteins include XylE: (from E. coli), QacA (from S. aureus), Bmr (of B. subtilis), UhpT (from E. coli), LacY (from E. coli), FucP (from E. coli), and ExtU (from E. coli).

In some embodiments, the transporter is of SWEET (Sugars Will Eventually be Exported Transporters) family of transport proteins (a/k/a the PQ-loop, Saliva or MtN3 family), which is a family of sugar transporters and a member of the TOG superfamily. Eukaryotic family members of SWEET have 7 transmembrane segments (TMSs) in a 3+1+3 repeat arrangement. By way of example, SWEET transporter proteins include SWEET1, SWEET2, SWEET9, SWEET12, SWEET13, and SWEET14.

In some embodiments, the the transport protein is at least 50% identical to a transport protein from S. cerevisiae. In some embodiments, the transporter is at least about 60%, at least about 70%, at least about 80%, at least about 90%, or at least about 95% identical to the S. cerevisiae transporter. Exemplary S. cerevisiae transport proteins include AC1, ADP1, ANTI, AQR1, AQY3, ARN1, ARN2, ARR3, ATG22, ATP4, ATP7, ATP19, ATR1, ATX2, AUS1, AVT3, AVT5, AVT6, AVT7, AZR1, CAF16, CCH1, COT1, CRC1, CTR3, DAL4, DNF1, DNF2, DTR1, DUR3, ECM3, ECM27, ENB1, ERS1, FEN2, FLR1, FSF1, FUR4, GAP1, GET3, GEX2, GGC1, GUP1, HOL1, HCT10, HXT3, HXT5, HXT8, HXT9, HXT11, HXT15, KHA1, ITR1, LEU5, LYP1, MCH1, MCH5, MDL2, MME1, MNR2, MPH2, MPH3, MRS2, MRS3, MTM1, MUP3, NFT1, OAC1, ODC2, OPT1, ORT1, PCA1, PDR1, PDR3, PDR5, PDR8, PDR10, PDR11, PDR12, PDR15, PDR18, PDRI, PDRI 1, PET8, PHO89, PIC2, PMA2, PMC1, PMR1, PRM10, PUT4, QDR1, QDR2, QDR3, RCH1, SAL1, SAM3, SBH2, SEO1, SGE1, SIT1, SLY41, SMF1, SNF3, SNQ2, SPF1, SRP101, SSU1, STE6, STL1, SUL1, TAT2, THI7, THI73, TIM8, TIM13, TOK1, TOM7, TOM70, TPN1, TPO1, TPO2, TPO3, TPO4, TRK2, UGA4, VBA3, VBA5, VCX1, VMA1, VMA3, VMA4, VMA6, VMR1, VPS73, YEA6, YHK8, YIA6, YMC1, YMD8, YOR1, YPK9, YVC1, ZRT1; YBR241C, YBR287W, YDR061W, YDR338C, YFR045W, YGL114W, YGR125W, YIL166C, YKL050C, YMR253C, YMR279C, YNL095C, YOL075C, YPR003C, and YPR011C.

In some embodiments, the S. cerevisiae transport protein is selected from one or more of ADP1, AQR1, ARN1, ARN2, ATR1, AUS1, AZR1, DAL4, DTR1, ENB1, FLR1, GEX2, HOL1, HXT3, HXT8, HXT11, NFT1, PDR1, PDR3, PDR5, PDR8, PDR10, PDR11, PDR12, PDR15, PDR18, QDR1, QDR2, QDR3, SEO1, SGE1, SIT1, SNQ2, SSU1, STE6, THI7, THI73, TIM8, TPN1, TPO1, TPO2, TPO3, TPO4, YHK8, YMD8, YOR1, and YVC1. In some embodiments, S. cerevisiae transport protein is selected from one or more of FLR1, PDR1, PDR3, PDR5, PDR10, PDR15, SNQ2, TPO1, and YOR1.

In some embodiments, the transporter is at least 50% identical to XP_013706116.1 (from Brassica napus), NP_001288941.1 (from Brassica rapa), NEC1 (from Petitnia hybrida ), and SWEET13 (from Triticum urartu).

The bacterial cell produces the target steviol glycosides through a plurality of uridine diphosphate dependent glycosyltransferase (UGT) enzymes. For example, the host cell further expresses a steviol glycoside enzymatic pathway, for the expression of the desired steviol glycoside, such as RebM. An enzymatic pathway for production of steviol glycosides, including RebM, is disclosed in WO 2016/073740, which is hereby incorporated by reference in its entirety. The pathway includes one or more UGT enzymes having glycosylating activity at C19 and C13 of steviol, and one or more UGT enzymes having 1-2′ and 1-3′ glycosylating activity at C19 and C13. Exemplary engineered UGT enzymes are listed in Tables 1 and 2 below.

TABLE 1

UGT enzymes for production of steviol glycosides

| Type of glycosylation | Enzyme | Gene ID | Protein ID | Description |
|---|---|---|---|---|
| C13 | SrUGT85C2 | AY345978.1 | AAR06916.1 | |
| C19 | SrUGT74G1 | AY 345982.1 | AAR06920.1 | |
| | MbUGTc19 | - | - | WO 2016/073740 |
| 1-2′ | SrUGT91D1 | AY345980.1 | AAR06918.1 | |
| | SrUGT91D2 | ACE87855.1 | ACE87855.1 | |
| | SrUGT91D2e | - | - | US 2011/038967 |
| | OsUGT1-2 | NM_001057542.1 | NP_001051007.2 | WO 2013/022989 |
| | MbUGT1-2 | - | - | WO 2016/073740 |
| 1-3′ | SrUGT76G1 | FB917645.1 | CAX02464.1 | |

TABLE 2

Enzymes known to catalyze reactions required for steviol glycoside biosynthesis

| Substrate | Product | Type of glycosylation | Enzyme 1 | Enzyme 2 | Enzyme 3 | Enzyme 4 |
|---|---|---|---|---|---|---|
| Steviol | Steviolmonoside | C13 | SrUGT85C2 | | | |
| Steviol | C19-Glu-Steviol | C19 | SrUGT74G1 | MbUGTc19 | | |
| Steviolmonoside | Steviolbioside | 1-2′ | SrUGT91D1 | SrUGT91D2 | OsUGT1-2 | MbUGT1-2 |
| Steviolmonoside | Rubusoside | C19 | SrUGT74G1 | MbUGTc19 | | |
| C19-Glu-Steviol | Rubusoside | C13 | SrUGT85C2 | | | |
| Steviolbioside | Stevioside | C19 | SrUGT74G1 | MbUGTc19 | | |
| Steviolbioside | RebB | 1-3′ | SrUGT76G1 | | | |
| Stevioside | RebE | 1-2′ | SrUGT91D1 | SrUGT91D2 | OsUGT1-2 | MbUGT1-2 |
| Stevioside | RebA | 1-3′ | SrUGT76G1 | | | |
| RebB | RebA | C19 | SrUGT74G1 | MbUGTc19 | | |
| RebE | RebD | 1-3′ | SrUGT76G1 | | | |
| RebA | RebD | 1-2′ | SrUGT91D1 | SrUGT91D2 | OsUGT1-2 | MbUGT1-2 |
| RebD | RebM | 1-3′ | SrUGT76G1 | | | |

The bacterial cell produces steviol substrate through an enzymatic pathway comprising a kaurene synthase (KS), kaurene oxidase (KO), and a kaurenoic acid hydroxylase (KAH). The host cell may further comprise a cytochrome P450 reductase (CPR) for regenerating one or more of the KO and KAH enzymes. The host cell may further express a geranylgeranyl pyrophosphate synthase to generate (GGPPS). Exemplary enzymes are disclosed in WO 2016/073740, which is hereby incorporated by reference. Exemplary enzymes are listed in Table 3.

TABLE 3

Summary of enzyme/gene sequences enabling biosynthesis of steviol.

| No. | Enzyme | Species | Gene ID | Protein ID |
|---|---|---|---|---|
| 1 | TcGGPPS | Taxus canadensis | AF081514.1 | AAD16018.1 |
| 2 | AgGGPPS | Abies grandis | AF425235.2 | AAL17614.2 |
| 3 | AnGGPPS | Aspergillus nidulans | XM_654104.1 | XP_659196.1 |
| 4 | SmGGPPS | Streptomyces melanosporofaciens | AB448947.1 | BAI44337.1 |
| 5 | MbGGPPS | Marine bacterium 443 | n/a | AAR37858.1 |
| 6 | PhGGPPS | Paracoccus haeundaensis | n/a | AAY28422.1 |
| 7 | CtGGPPS | Chlorobium tepidum TLS | NC_002932.3 | NP_661160.1 |
| 8 | SsGGPPS | Synechococcus sp. JA-3-3Ab | n/a | ABC98596.1 |
| 9 | Ss2GGPPS | Synechocystis sp. PCC 6803 | n/a | BAA16690.1 |
| 10 | TmGGPPS | Thermotoga maritima HB8 | n/a | NP_227976.1 |
| 11 | CgGGPPS | Corynebacterium glutamicum | n/a | NP_601376.2 |
| 12 | TtGGPPS | Thermus thermophillus HB27 | n/a | YP_143279.1 |
| 13 | PcGGPPS | Pyrobaculum calidifontis JCM 11548 | n/a | WP_011848845.1 |
| 14 | SrCPPS | Stevia rebaudiana | AF034545.1 | AAB87091.1 |
| 15 | EtCPPS | Erwina tracheiphila | n/a | WP_020322919.1 |
| 16 | SfCPPS | Sinorhizobium fredii | n/a | WP_010875301.1 |
| 17 | SrKS | Stevia rebaudiana | AF097311.1 | AAD34295.1 |
| 18 | EtKS | Erwina tracheiphila | n/a | WP_020322918.1 |
| 19 | SfKS | Sinorhizobium fredii | n/a | WP_010875302.1 |
| 20 | GfCPPS/KS | Gibberella fujikuroi | AB013295.1 | Q9UVY5.1 |
| 21 | PpCPPS/KS | Physcomitrella patens | AB302933.1 | BAF61135.1 |
| 22 | PsCPPS/KS | Phaeosphaeria sp. L487 | AB003395.1 | 013284.1 |
| 23 | AtKO | Arabidopsis thaliana | NM_122491.2 | NP_197962.1 |
| 24 | SrKO | Stevia rebaudiana | AY364317.1 | AAO63464.1 |
| 25 | PpKO | Physcomitrella patens | AB618673.1 | BAK19917.1 |
| 26 | AtCPR | Arabidopsis thaliana | X66016.1 | CAA46814.1 |
| 27 | SrCPR | Stevia rebaudiana | DQ269454.4 | ABB88839.2 |
| 28 | AtKAH | Arabidopsis thaliana | NM_122399.2 | NP_197872.1 |
| 29 | SrKAH1 | Stevia rebaudiana | DQ398871.3 | ABD60225.1 |
| 30 | SrKAH2 | Stevia rebaudiana | n/a | n/a |

In some embodiments, the host cell expresses a pathway producing iso-pentyl pyrophosphate (IPP) and dimethylallyl pyrophosphate (DMAPP), such as a bacterial strain. In some embodiments, the pathway is a methylerythritol phosphate (MEP) pathway.

The MEP (2-C-methyl-D-erythritol 4-phosphate) pathway, also called the MEP/DOXP (2-C-methyl-D-erythritol 4-phosphate/l-deoxy-D-xylulose 5-phosphate) pathway or the non-mevalonate pathway or the mevalonic acid-independent pathway refers to the pathway that converts glyceraldehyde-3-phosphate and pyruvate to IPP and DMAPP. The pathway typically involves action of the following enzymes: 1-deoxy-D-xylulose-5-phosphate synthase (Dxs), 1-deoxy-D-xylulose-5-phosphate reductoisomerase (IspC), 4-diphosphocytidyl-2-C-methyl-D-erythritol synthase (IspD), 4-diphosphocytidyl-2-C-methyl-D-erythritol kinase (IspE), 2C-methyl-D-erythritol 2,4-cyclodiphosphate synthase (IspF), 1-hydroxy-2-methyl-2-(E)-butenyl 4-diphosphate synthase (IspG), and isopentenyl diphosphate isomerase (IspH). The MEP pathway, and the genes and enzymes that make up the MEP pathway, are described in US 8,512,988, which is hereby incorporated by reference in its entirety. For example, genes that make up the MEP pathway include dxs, ispC, ispD, ispE, ispF, ispG, ispH, idi, and ispA. In some embodiments, steviol is produced at least in part by metabolic flux through an MEP pathway, and wherein the host cell has at least one additional copy of a dxs, ispD, ispF, and/or idi gene. As disclosed in US 8,512,988, the level of the metabolite indole can be used as a surrogate marker for efficient production of terpenoid products in E. coli through the MEP pathway.

In some embodiments, the host strain is a bacterial strain with improved carbon flux into the MEP pathway and to a downstream recombinant synthesis pathway thereby increasing steviol glycoside production by fermentation with inexpensive carbon sources (e.g., glucose).

In some embodiments, the bacterial strain overexpresses IspG and IspH, so as to provide increased carbon flux to 1-hydroxy-2-methyl-2-(E)-butenyl 4-diphosphate (HMBPP) intermediate, but with balanced expression to prevent accumulation of HMBPP at an amount that reduces cell growth or viability. Increasing expression of both IspG and IspH significantly increases titers of terpene and terpenoid products. In contrast, overexpression of IspG alone results in growth defects, while overexpression of IspH alone does not significantly impact product titer. See US 62/450,707, which is hereby incorporated by reference in its entirety.

In various embodiments, the bacterial strain overexpresses a balanced MEP pathway to move MEP carbon to the MEcPP intermediate, the substrate for IspG, and includes one or more modifications to support the activities of IspG and IspH enzymes, which are Fe-sulfur cluster enzymes. In certain embodiments, the bacterial strain contains an inactivation or deletion of fnr to maintain aerobic metabolism. See US 62/450,707, which is hereby incorporated by reference in its entirety.

In some embodiments, the target steviol glycoside is produced in the culture media at a concentration of at least about 100 mg/L, or at least about 200 mg/L, or at least about 500 mg/L, or at least about 1 g/L, or at least about 10 g/L.

In some embodiments, the method further comprises separating or purifying one or more target steviol glycosides. In some embodiments, the target steviol glycoside can be separated by any suitable method known in the art, such as, for example, crystallization, separation by membranes, centrifugation, extraction, chromatographic separation or a combination of such methods. In some embodiments, limited purification steps are required, since the host cells produce the desired steviol glycoside almost exclusively in the culture medium.

In some embodiments, the culturing is conducted at about 30° C. or greater, or about 31° C. or greater, or about 32° C. or greater, or about 33° C. or greater, or about 34° C. or greater, or about 35° C. or greater, or about 36° C. or greater, or about 37° C.

In some embodiments, the engineered host cells and methods disclosed herein are suitable for commercial production of steviol glycosides, that is, the cells and methods can be productive at commercial scale. In some embodiments, the size of the culture is at least about 100 L, at least about 200 L, at least about 500 L, at least about 1,000 L, at least about 10,000 L, or at least about 100,000 L. In an embodiment, the culturing may be conducted in batch culture, continuous culture, or semi-continuous culture.

In some embodiments, the present technology further provides methods of making products containing steviol glycosides, e.g., RebM, including food products, beverages, oral care products, sweeteners, flavoring products, among others. Such steviol glycoside-containing products are produced at reduced cost by virtue of this disclosure. In some embodiments, the present technology provides methods for making a product comprising one or more steviol glycosides, e.g., RebM or RebD. In some embodiments, the method comprises culturing one or more engineered host cells disclosed herein under conditions that produce one or more steviol glycosides, recovering the one or more steviol glycosides, and incorporating one or more steviol glycosides into a product. In some embodiments, the product is selected from a food, beverage, oral care product, sweetener, flavoring agent, or other product. In some embodiments, RebM is the steviol glyocide recovered and incorporated into the product.

In some embodiments, the one or more recovered or purified steviol glycosides, prepared in accordance with the present technology, is used in a variety of products including, but not limited to, foods, beverages, texturants (e.g., starches, fibers, gums, fats and fat mimetics, and emulsifiers), pharmaceutical compositions, tobacco products, nutraceutical compositions, oral hygiene compositions, and cosmetic compositions. Non-limiting examples of flavors for which RebM can be used in combination include lime, lemon, orange, fruit, banana, grape, pear, pineapple, mango, bitter almond, cola, cinnamon, sugar, cotton candy and vanilla flavors. Non-limiting examples of other food ingredients include flavors, acidulants, and amino acids, coloring agents, bulking agents, modified starches, gums, texturizers, preservatives, antioxidants, emulsifiers, stabilizers, thickeners and gelling agents.

All patents, patent applications, provisional applications, and publications referred to or cited herein are incorporated by reference in their entirety, including all figures and tables, to the extent they are not inconsistent with the explicit teachings of this specification.

1. A method for making a target steviol glycoside composition, comprising:
culturing an engineered microbial cell producing one or more target steviol glycosides, wherein the engineered microbial cell comprises recombinant expression of one or more transport proteins that transport the target steviol glycosides into the extracellular medium, and
recovering the target steviol glycosides from the extracellular medium.

2. The method of claim 1, wherein the cell is a bacterial cell.

3. The method of claim 1 , wherein the target steviol glycoside in RebM.

4. The method of claim 1, wherein the target steviol glycoside includes one or more selected from steviolmonoside, steviolbioside, rubusoside, dulcoside B, dulcoside A, stevioside, rebaudioside A (RebA), rebaudioside B (RebB), rebaudioside C (RebC), rebaudioside D (RebD), rebaudioside D2 (RebD2), rebaudioside E (RebE), rebaudioside F (RebF), rebaudioside G (RebG), rebaudioside H (RebH), rebaudioside I (RebI), rebaudioside J (RebJ), rebaudioside K (RebK), rebaudioside L (RebL), rebaudioside M (RebM), rebaudioside M2 (RebM2), rebaudioside N (RebN), and rebaudioside O (RebO).

5-6. (canceled)

7. The method of claim 2, wherein the bacterial species is E. coli.

8. The method of claim 1, wherein the host cell contains a deletion or inactivaction of one or more endogenous transporters that transport a steviol glycoside other than a target steviol glycoside.

9. The method of claim 1, wherein the host cell overexpresses one or more endogeous transport proteins that transport the target steviol glycoside(s).

10-21. (canceled)

22. The method of claim 1, wherein the host cell produces the target steviol glycosides through a plurality of uridine diphosphate dependent glycosyltransferase (UGT) enzymes.

23. The method of claim 1, wherein the host cell produces steviol substrate through an enzymatic pathway comprising a kaurene synthase (KS), kaurene oxidase (KO), and a kaurenoic acid hydroxylase (KAH).

24. The method of claim 1, wherein the host cell overexpresses one or more enzymes of the MEP pathway, producing iso-pentyl pyrophosphate (IPP) and dimethylallyl pyrophosphate (DMAPP).

25. An engineered host cell producing one or more target steviol glycosides, wherein the engineered bacterial cell comprises recombinant expression of one or more transport proteins that transport the target steviol glycosides into the extracellular medium.

26. The host cell of claim 25, wherein the host cell is a bacterial cell.

27. The host cell of claim 25, wherein the target steviol glycoside in RebM.

28. The host cell of claim 25, wherein the target steviol glycoside includes one or more selected from steviolmonoside, steviolbioside, rubusoside, dulcoside B, dulcoside A, stevioside, rebaudioside A (RebA), rebaudioside B (RebB), rebaudioside C (RebC), rebaudioside D (RebD), rebaudioside D2 (RebD2), rebaudioside E (RebE), rebaudioside F (RebF), rebaudioside G (RebG), rebaudioside H (RebH), rebaudioside I (RebI), rebaudioside J (RebJ), rebaudioside K (RebK), rebaudioside L (RebL), rebaudioside M (RebM), rebaudioside M2 (RebM2), rebaudioside N (RebN), and rebaudioside O (RebO).

29-30. (canceled)

31. The host cell of claim 26, wherein the bacterial species is E. coli.

32. The host cell of claim 25, wherein the host cell contains a deletion or inactivaction of one or more endogenous transporters that transport a steviol glycoside other than a target steviol glycoside.

33. The host cell of claim 25, wherein the host cell overexpresses one or more endogeous transport proteins that transport the target steviol glycoside(s).

34-45. (canceled)

46. The host cell of claim 25, wherein the host cell produces the target steviol glycosides through a plurality of uridine diphosphate dependent glycosyltransferase (UGT) enzymes.

47. The host cell of claim 25 , wherein the host cell produces steviol substrate through an enzymatic pathway comprising a kaurene synthase (KS), kaurene oxidase (KO), and a kaurenoic acid hydroxylase (KAH).

48. The host cell of claim 25, wherein the host cell overexpresses one or more enzymes of the MEP pathway, producing iso-pentyl pyrophosphate (IPP) and dimethylallyl pyrophosphate (DMAPP).
